(19) United States
(12) Patent Application Publication
(10) Pub. No.: US 2005/0024743 A1
(43) Pub. Date: Feb. 3, 2005

(54) Focusing optic for laser cutting

(76) Inventor: Frederic Camy-Peyret, Paris (FR)

Correspondence Address: Air Liquide;Suite 1800, 2700 Post Oak Blvd., Houston, TX, 77056, US

(21) Appl. No.: 10/851,431

(22) Filed: May 21, 2004

(30) Foreign Application Priority Data
May 22, 2003 (FR) 0306155

(51) Int. Cl. B23K 26/06; B23K 26/38; B23K 26/14; G02B 3/02; G02B 13/18; A61B 18/18

(52) U.S. Cl. 359/719; 219/121.72

(57) ABSTRACT

The invention relates to an optical component (21), which can be used for the laser cutting of a material (26), comprising at least one aspherical refractive surface (22) shaped to focus the rays (30) of the incident beam onto a straight line segment (25) lying on the optical axis (29) of the optical component (21). The optical component (21) is of the transmissive type (21) or reflective type. For example, the optical element is formed by a lens (21) whose aspherical refractive surface (22) is defined by a radius of curvature (24) that varies continuously with the distance from the optical axis (29) of the lens. The laser beam is assisted by an assistance gas containing at least one component chosen from nitrogen, oxygen, helium, argon and mixtures thereof.

The present invention relates to an optical component, such as a particular aspherical lens, that can be used for the laser cutting of a material, in particular of metals or metal alloys, and to a laser cutting process using such an optical component.

Laser beam cutting is a process for cutting materials, particularly metals or metal alloys, widely used in industry.

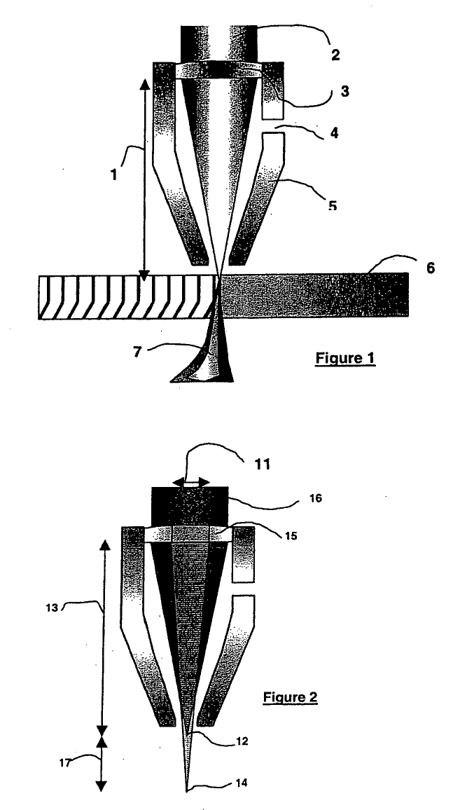

In short, the use of this process involves, as shown schematically in FIG. 1, a laser beam 2, for example output by a CO₂ laser (λ=10.6 μm) or an Nd:YAG (λ=1.06 μm) laser, focused by at least one optical element 3, of the lens or mirror type, of given focal length 1, onto a workpiece 6 to be cut.

An assistance gas is injected into the cutting kerf so as to remove the molten metal 7, the said kerf being created by relative movement of the cutting head with respect to the workpiece 6 to be cut.

The cutting head comprises the optical focusing component 3 and a cutting nozzle 5 provided with at least one gas inlet 4 for injecting the cutting gas into the nozzle 5.

The gas introduced into the cutting head 5 emerges therefrom via one or more ejection channels or orifices that face the workpiece 6 to be cut, the laser beam 2, focused upstream by the focusing optic 3, generally also passing through one of the said channels or orifices.

Various forms of assistance-gas ejection orifices, such as Laval nozzles or convergent-divergent nozzles of minimum length, and also nozzles with coalescent jets and dual-gas-flow devices, may be used to improve the performance.

Transmissive focusing optics, that is to say optical lenses, are the optical components most widely used for laser cutting in that they create a pressure-tight cavity in the cutting head into which the assistance gas can be injected and then exit via a nozzle 5 coaxial with the laser beam.

A focusing lens has two refractive surfaces, that is to say two faces, on which an anti-reflection treatment or coating is deposited so as to limit loss of power by reflection.

The material of the lens core is often made of zinc selenide (ZnSe) for CO₂ lasers and “bk7”-type glass for Nd:YAG lasers.

The various forms of lens mainly used in the industry are:

plano-convex lenses composed of a spherical refractive surface and a plane refractive surface;

meniscus lenses composed of two spherical refractive surfaces. Since this shape has the advantage of minimizing spherical aberrations compared with plano-convex lenses, they are very widely used in laser cutting; and

aspherical lenses for which the shape of the first refractive surface of these lenses, which is no longer a sphere of constant radius, is optimized so as to further reduce geometrical aberrations compared with a meniscus lens with spherical refractive surfaces and thus to obtain higher power densities at the focal point, in particular in the case of short focal lengths, that is to say those less than 95.25 mm (3.75″). The exit refractive surface of aspherical lenses is generally plane, this being mainly to reduce their manufacturing cost.

All these lenses tend to focus the laser beam onto a single focal point of minimal size.

However, a concept has been presented in Document WO 98/14302 that is based on optics with several focal points that improve the performance of the laser cutting process.

The shape of these optics, whether of the lens or mirror type, is such that the incident beam is no longer focused at a single point but at two or more focal points, as shown schematically in FIG. 2.

According to that document, when a dual-focus lens 15 is used, that portion of the incident beam 16 located on the outside, with a diameter 11, is focused at a first focal point 12 corresponding to a main focal length 13, whereas that portion of the incident beam being located on the inside, with a diameter 11, is focused at a second focal point 14 located at a distance 17 beyond the first focal point 12 in the direction of light propagation of the beam.

This dual-focus lens is produced with a radius of curvature of one of the refractive surfaces, that of the convex face for example, which is different inside the diameter 11 from that outside the diameter 11.

This type of focusing optic makes it possible to achieve increases in cutting speed and/or quality, or even in tolerance of the process with respect to variations in the distance between the lens and the workpiece, and/or in ability to cut thicker materials than conventional lenses with a single focal point.

However, the characteristics of the power density field given by a bifocal lens remain limited by the discrete choice of the number of focal points.

Since the radii of curvature of the first refractive surface are constant in intervals, current bifocal or multifocal lenses and mirrors do not allow optimized adjustment of the lens to the characteristics of the beam and to the customer's application.

The increases in productivity obtained compared with monofocal lenses, that is to say conventional lenses in FIG. 1, are due to a distribution in the power and power density in the cutting kerf that present two or more maxima along the optical axis, but this energy distribution is not optimal as it is not continuous over the entire thickness of the workpiece.

Moreover, the bifocal or multifocal optics used in laser cutting are sensitive to variations in the diameter of the beam, since the power distribution between the various focal points depends on the diameter of the beam.

Monofocal optics are also sensitive to beam variations, since a change in divergence of the incident beam can cause a change in the position of the focal point. This makes the process less tolerant and causes difficulties in keeping the cutting quality constant, for example when the cutting head moves and when the length of the optical path between the laser and the head varies.

The problem that then arises is to propose an improved focusing optic so as to allow, when it is used in a laser beam cutting process, better distribution of the energy delivered into the workpiece to be cut, that is to say into the cutting kerf, and therefore also to increase productivity compared with conventional monofocal and multifocal optics.

The solution of the invention is therefore an optical component, which can be used for the laser cutting of a material, comprising at least one aspherical refractive surface shaped to focus the rays of the incident beam onto a straight line segment lying on the optical axis of the optical component.

Within the context of the invention:

the expression “straight line segment” is understood to mean that the laser beam is focused in a region consisting of an infinity of points aligned so as to form a continuous linear focusing region, that is to say a portion of a straight line, with a length that may range from 0.01 mm to 50 mm, bounded by two points at its ends;

the term “optical axis” is understood to mean the axis of symmetry of the lens and that of the incident laser beam, these generally being in alignment and forming a single straight line in space, called the optical axis.

Depending on the case, the optical component of the invention may include one or more of the technical features given below:

it is of the transmissive or reflective type;

the straight line segment onto which the beam is focused has a length of 0.01 to 50 mm, preferably around 1 to 20 mm, depending on the thickness and the nature of the material;

it is formed by a lens whose aspherical refractive surface (i.e. on the incident side, that is to say the refractive surface that is first impacted by the beam) is defined by a radius of curvature that varies continually with the distance from the optical axis of the lens;

it is formed by a lens whose exit refractive surface is plane; and

it is formed by a lens having a diameter between 4 mm and 60 mm.

The invention also relates to a process for the laser beam cutting of a material, in which at least one optical component according to the invention is used to focus the said laser beam.

Depending on the case, the process of the invention may include one or more of the technical features given below:

the optical component focuses the laser beam onto a straight line segment lying on the optical axis of the said optical component and within the thickness of the material to be cut;

the straight line segment has a length equal or approximately equal to the thickness of the material to be cut;

the ray of the incident beam arriving at the centre of the lens is focused close to the underside of the material to be cut and the ray arriving at the periphery of the lens is focused close to the top side of the material to be cut. In this way, it is possible to achieve focusing over the entire thickness of the material to be cut, the straight line segment onto which the rays of the laser beam are focused then being coincident with the axis of the optical component and has a length equal to the entire thickness of the workpiece to be cut;

the locus of the points of intersection of the rays of the incident beam with the optical axis forms a focusing segment, the energy of the laser beam being distributed continuously along the said segment; and

the laser beam is assisted by means of an assistance gas containing at least one component chosen from nitrogen, oxygen, helium, argon and mixtures thereof, for example binary, ternary, quaternary or other mixtures, such as nitrogen/oxygen mixtures, argon/helium mixtures, nitrogen/helium mixtures, nitrogen/argon/oxygen mixtures, etc., which may also include other constituents, in particular hydrogen, CO₂, etc., the said gas to be used being chosen according to the nature of the metal or alloy to be cut.

In other words, the invention relates to an optical component for the laser cutting of materials that may be transmissive, such as a lens, or reflective, such as a mirror, and which has at least one aspherical refractive surface that focuses the rays of the incident beam onto a straight line segment lying on the optical axis.

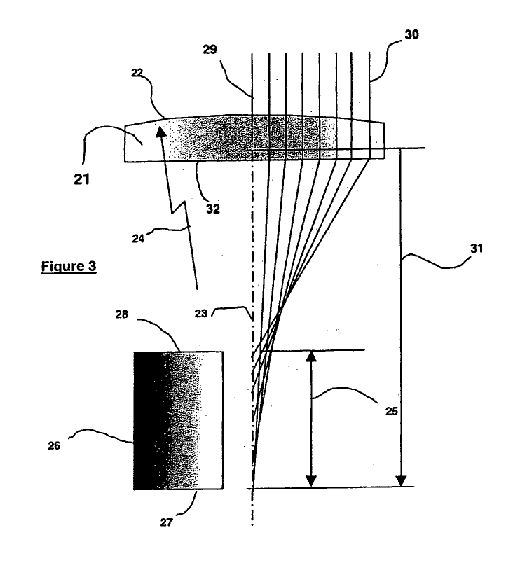

In the case of a transmissive optic, such as the lens 21 shown schematically in FIG. 3, the refractive surface 22 is defined by a radius of curvature 24 that varies continuously with the distance from the optical axis 29 of the lens so as to focus the incident rays arriving on the lens onto a straight line segment 25 lying on the optical axis 29 of the lens.

This segment may have a length close to the thickness of the material 26 to be cut. To do this, the ray 29 of the incident beam arriving at the centre of the lens may advantageously be focused close to the underside 27 of the component to be cut and the ray 30 arriving at the periphery of the lens may be focused close to the top side 28 of the workpiece to be cut.

The locus of the points of intersection of the rays of the incident beam with the optical axis forms a focusing segment 25 along which the energy of the beam is distributed continuously.

The exit refractive surface 32 of the lens shown in the example of FIG. 3 may be plane in order to reduce the manufacturing costs.

It should be noted that the linear-focusing optic according to the present invention differs from the known optics with aspherical refractive surfaces in that the objective pursued is not to focus the beam onto an area as small as possible, limited only by the diffraction or by the quality of the beam, but to distribute the incident beam continuously along a focusing segment lying on the optical axis.

The distribution of the energy delivered into the workpiece to be cut is in this way better distributed within the kerf and makes it possible to achieve increased productivity compared with: monofocal and multifocal optics.

Likewise, the present invention also differs from the multifocal optics presented in Document WO-A-98/14302 in that it generates a focusing segment along which the intensity of the laser beam is distributed continuously, and not a discrete number of successive focal points.

According to the invention, the distribution of the incident beam along the focusing segment is determined by the shape of aspherical refractive surface and in particular by the continuous function that defines its radius of curvature as a function of the distance from the optical axis. This function may be tailored to the thickness and to the nature of the material to be cut, and also to the distribution profile of the light power density of the incident beam.

In particular, it is possible to define this aspherical refractive surface in such a way that the radius of curvature of the refractive surface is a function of the radial distribution of the power density of the incident beam so as to obtain:

a uniform power density along the focusing segment; or

a power density along the focusing segment that has a maximum close to both the top side and the underside of the workpiece.

The laser beams used in the industry are frequently of variable diameter and variable divergence. In particular, in the case of moving focusing heads, the length of the optical path and therefore the diameter and divergence of the beam depend on the position of the head on the cutting table.

An advantage of the optic of the invention shown schematically in FIG. 3 is that the variations in beam diameter have less influence on the power distribution and power density distribution in the cutting kerf than the known bifocal or multifocal systems.

This is because a variation in beam diameter causes a continuous variation in the function defining the power distribution along the focusing segment and provides greater tolerance to this variable.

Variations in the divergence of the incident beam also have less influence on the quality of the cutting than in the case of monofocal lenses.

This is because, since the energy is continuously distributed over a vertical segment, the shift along the optical axis of this focusing segment relative to the workpiece when the divergence of the incident beam varies has less impact on the power density transmitted to the workpiece than when the energy is concentrated at a single focal point, the position of which relative to the workpiece is a key parameter for obtaining good performance.

The use of an optic with a progressive radius of curvature according to the invention therefore makes it possible to achieve further increases in productivity, for example in cutting speed, up to the limits of the cutting process without any fear of a sudden drop in cutting quality, or even a complete loss of cutting as happens when monofocal, and to a lesser extent bifocal or multifocal, optics are used.

In general, the optical component of the invention may be formed by a lens 21 whose aspherical refractive surface 22 is defined by an equation that includes a term logarhithmically dependent on the distance from the optical axis 29, for example, but not limiting, by the equation B²Cr=AlnA+Bz−Aln(A+Bz) where the (r,z) pairs that satisfy the equation form the set of coordinates of the points defining the refractive surface in a reference frame of orthonormal axes (\vec{r},\vec{z}), where \vec{r} is the radial unit vector perpendicular to the optical axis, where \vec{z} is the axial unit vector collinear to the optical axis and where A, B and C are constants dependent on the incident beam, the material and the application.

Alternatively, the optical component of the invention may also be formed by a lens 21 whose aspherical refractive surface 22 is defined by the equation of a conic, for example, but not limitingly, by the equation r²+Pz²−2Rz=0 where the (r,z) pairs satisfying the equation form the set of coordinates of the points defining the refractive surface in a reference frame of orthonormal axes (\vec{r},\vec{z}), where \vec{r} is the radial unit vector perpendicular to the optical axis, where \vec{z} is the axial unit vector collinear with the optical axis and where P and R are constants dependent on the incident beam, the material and the application.

In both cases, numerical values solving one or other of the above equations are chosen in such a way that the aspherical refractive surface results in focusing along a continuous segment according to the invention.

Within the context of the invention, the gases or mixtures given in the following table may be used for cutting the materials indicated, especially for the purpose of obtaining a positive effect on the cutting speed or the quality of the cutting.

TABLE
material to be cut/gas combinations

| Materials | Oxygen | Nitrogen | Argon | Helium |
|---|---|---|---|---|
| Low-alloy | YES/speed | YES/quality | Possible | Possible |
| steels | | | | |
| Stainless | YES/speed | YES/quality | Possible | Possible |
| steels | | | | |
| Aluminium | YES/speed | YES/quality | Possible | Possible |
| alloys | | | | |
| Nickel | YES/speed | YES/quality | Possible | Possible |
| alloys | | | | |
| Titanium | Not | Not | YES/quality | YES/ |
| alloys | recommended | recommended | | quality |

Of course, certain gas mixtures could also be used instead of the gases listed in the above table so as to take advantage of the properties of the components of the mixture thus obtained. For example, to cut a stainless steel, it is possible to use an oxygen/nitrogen mixture when it is desired to increase both cutting speed and quality compared with nitrogen alone or with oxygen alone.

Likewise, the gases given in the above table may be combined with other gaseous compounds, the action of which may be beneficial for cutting a particular material. For example, nitrogen/argon mixtures to which hydrogen has been added (to less than 30 vol %) could be used for cutting stainless steel so as to obtain burr-free and shiny cut faces (no oxides deposited), that is to say high-quality cut faces.

Within the context of the invention, all the various methods of distributing the assistance gas that are used to improve the cutting performance and described above may be used.

### ILLUSTRATIVE EXAMPLE

In the following example, a lens according to the present invention was used to cut a 6 mm thick aluminium plate of AUG4 grade with a CO₂ laser beam of 4 kW power, the transverse intensity distribution mode (00 electromagnetic transverse mode) was Gaussian with a 14 mm diameter incident on the lens at 86% power.

The lens had a plane exit refractive surface and an aspherical entry refractive surface, the latter being an ellipsoid of revolution focusing the incident beam on a straight line segment around 5 mm in length.

The lower end of this segment was approximately 127 mm from the exit refractive surface of the lens, which had a diameter of approximately 38.1 mm and a thickness at the edges of about 7.6 mm.

The faces of the lenses were coated with an anti-reflection treatment in accordance with the prior art.

The gas used for the cutting was nitrogen injected at a relative pressure of 15 bar into a 2 mm diameter nozzle.

The use of this lens, compared with that of a conventional monofocal lens of 190 mm focal length, made it possible to achieve a cutting speed of around 2.4 m/min, an increase of approximately 33% over the 1.8 m/min speed obtained with a monofocal lens.

Compared with a bifocal lens of 38.1 mm outside diameter (focusing the beam at two separate points spaced apart), with a main focal length of 190 mm and a distance of 7.5 mm between the two focal points, for which the cutting speed was 2.15 m/min, the increase in speed was about 12%.

1-12. (cancelled).

13. An optical component apparatus, which may be used for the laser cutting of a material, comprising at least one aspherical refractive surface shaped to focus the rays of an incident beam onto a straight line segment lying on the optical axis of the optical component.

14. The apparatus of claim 13, wherein the type of said component is transmissive or reflective.

15. The apparatus of claim 13, wherein said straight line segment has a length from about 0.01 to about 50 mm.

16. The apparatus of claim 15, wherein said length is from about 1 to about 20 mm.

17. The apparatus of claim 13, further comprising a lens whose aspherical refractive surface is defined by a radius of curvature that varies continuously with the distance from the optical axis of said lens.

18. The apparatus of claim 13, further comprising a lens whose exit refractive surface is plane.

19. The apparatus of claim 13, further comprising a lens having a diameter between about 4 mm and about 60 mm.

20. A process for the laser cutting of a material, comprising focusing the laser beam with at least one optical component, wherein said component comprises at least one aspherical refractive surface shaped to focus the rays of an incident beam onto a straight line segment lying on the optical axis of said optical component.

21. The process of claim 20, wherein said optical component focuses said laser beam onto said straight line segment lying on the optical axis of said optical component and within the thickness of the material to be cut.

22. The process of claim 20, wherein said straight line segment has a length equal or approximately equal to the thickness of the material to be cut.

23. The process of claim 20, wherein the ray of the incident beam arriving at the center of the lens is focused close to the underside of the material to be cut and the ray arriving at the periphery of the lens is focused close to the top side of the material to be cut.

24. The process of claim 20, wherein the locus of the points of intersection of the rays of the incident beam with the optical axis forms a focusing segment, thus resulting in the energy of said laser beam being distributed continuously along said segment.

25. The process of claim 20, wherein said laser beam is assisted by means of an assistance gas comprising at least one member selected from group consisting of:
a) nitrogen;
b) oxygen;
c) helium; and
d) argon.

26. A process for the laser cutting of a material, comprising focusing the laser beam with at least one optical component, wherein said component comprises at least one aspherical refractive surface shaped to focus the rays of an incident beam onto a straight line segment lying on the optical axis of said optical component, wherein said straight line segment has a length equal or approximately equal to the thickness of the material to be cut, and wherein said laser beam is assisted by means of an assistance gas comprising at least one member selected from group consisting of:
a) nitrogen;
b) oxygen;
c) helium; and
d) argon.
